(19) United States
(12) Patent Application Publication
(10) Pub. No.: US 2003/0225434 A1
(43) Pub. Date: Dec. 4, 2003

(54) Microcatheter

(76) Inventors: Jerald Glantz, Lake Elmo, MN (US); Daniel J. Vreeman, Rogers, MN (US); Nathan T. Lee, Golden Valley, MN (US); Victor R. Blackledge, Cologne, MN (US)

Correspondence Address: MERCHANT & GOULD PC, P.O. BOX 2903, MINNEAPOLIS, MN, 55402-0903, US

(21) Appl. No.: 10/160,561

(22) Filed: May 30, 2002

(51) Int. Cl. A61M 29/00

(52) U.S. Cl. 606/194; 604/96.01

(57) ABSTRACT

The present disclosure relates to a catheter having an elongate body defining a lumen. An angioplasty balloon is mounted adjacent a distal end of the elongate body. A flexible tip is located proximal to the angioplasty balloon. In one embodiment, the flexible tip is integral with the elongate body.

This invention was made with Government support under Small Business Independent Research Grant HL60320, awarded by the National Institutes of Health. The Government has certain rights in the invention.

### TECHNICAL FIELD

This invention pertains to a catheter device. More particularly, this invention pertains to microcatheters adapted to navigate within narrow vessels such as cerebral vessels.

### BACKGROUND

Strokes are the leading cause of disability among adults in the United States, and are the third leading cause of death. A stroke occurs when blood flow to one or more regions of the brain is interrupted causing brain cells to die. The extent of damage is dependent upon the part of the brain affected. Common symptoms of a stroke include loss of mental capacity, changes in vision or speech, loss of muscle control and coordination, dizziness, loss of sensation and weakness.

A stroke is most commonly caused by atherosclerosis. Atherosclerosis is the accumulation of plaque (i.e., fatty deposits) within blood vessels. A stroke occurs when plaque accumulates within a cerebral vessel to the extent that blood flow is substantially blocked. Blood clots that form in the brain, or emboli that become lodged in vessels of the brain, can also block blood flow to portions of the brain thereby causing a stroke. Occasionally, a stroke is caused by bleeding within the brain (e.g., rupture of a cerebral aneurysm).

Catheters have been developed for diagnosing and treating strokes. Since cerebral vessels are small in diameter, catheters for accessing cerebral vessels typically are relatively flexible and small in diameter. This type of catheter is often referred to as a “microcatheter.” Example microcatheters are disclosed in U.S. Pat. Nos. 5,919,171; 6,296,631; and 6,306,124.

### SUMMARY

The present disclosure relates generally to catheters and catheter systems. The disclosure provides examples of different inventive concepts that may be used separately or in combination with one another.

### BRIEF DESCRIPTION OF THE DRAWINGS

FIG. 1 illustrates a catheter that is an embodiment of one or more aspects of the present invention;

FIG. 2 is an enlarged view of a tip section of the catheter of FIG. 1;

FIG. 3 is an enlarged view of a balloon section of the catheter of FIG. 1;

FIG. 4 is an enlarged view of an inflation hub section of the catheter of FIG. 1;

FIG. 5 is an illustration of a vascular system of a human brain with a guidewire inserted within the vasculature;

FIG. 6 is an enlarged view of a treatment area of the brain of FIG. 5, a distal end of the guidewire is shown adjacent to the treatment area;

FIG. 7 illustrates the treatment area of FIG. 6 with a guide catheter placed over the guidewire;

FIG. 8 illustrates the treatment area of FIG. 6 with the guidewire removed from the guide catheter thereby leaving only the guide catheter;

FIG. 9 illustrates the treatment area of FIG. 8 with a balloon catheter inserted into the guide catheter;

FIG. 10 illustrates the treatment area of FIG. 9 with the balloon expanded to open an occlusion in the depicted vessel; and

FIG. 11 illustrates the vessel of FIG. 10 after the occlusion has been opened.

### DETAILED DESCRIPTION

With reference now the various drawing figures in which identical elements are numbered identically throughout, a description of a preferred embodiment will now be provided. The embodiment illustrates examples of numerous inventive aspects. The inventive aspects are not intended to be limited to the specific examples shown and described herein, and can be practiced in other configurations as will be appreciated by one of skill in the art.

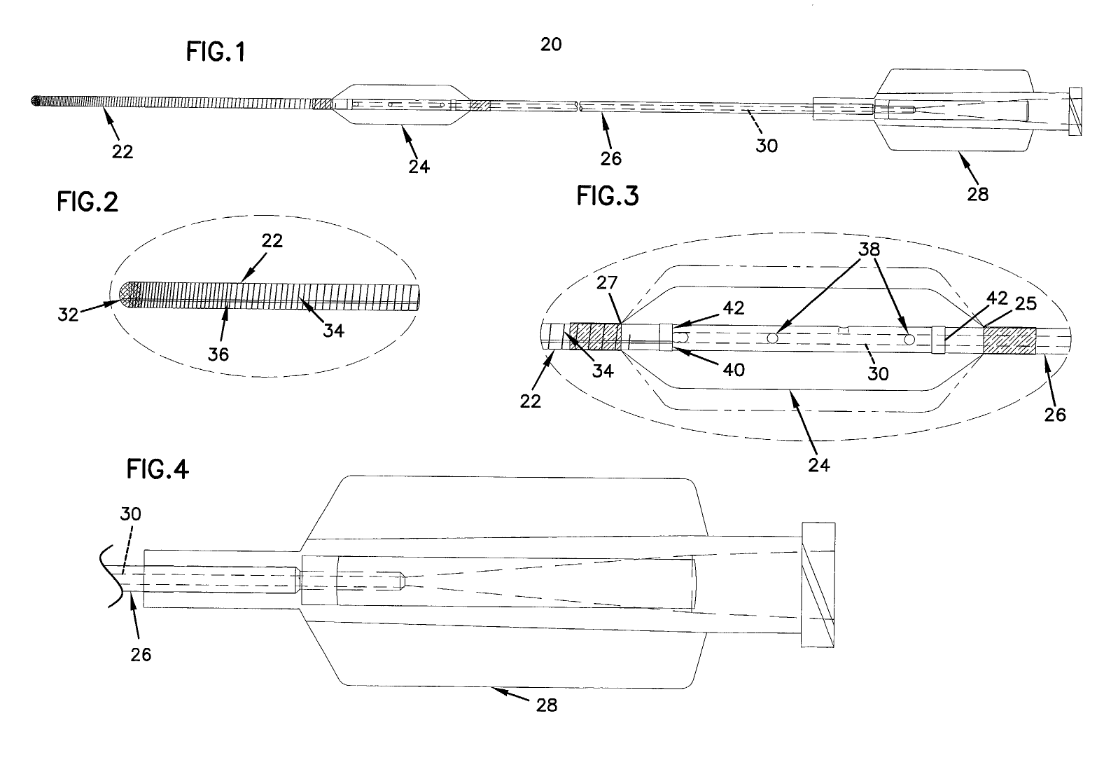

FIG. 1 shows an example of a catheter 20 made in accordance with the teachings of the present inventions. The catheter 20 includes a flexible distal tip 22, a balloon 24, an elongate body 26 and an inflation hub 28. The flexible distal tip 22 helps guide the catheter through tortuous vessels throughout the body or through other catheters (e.g., guide catheters). The balloon 24 can be used open occluded vessels. The elongate body 26 allows the user to maneuver the distal tip 22 and defines a lumen 30 that traverses the elongate body 26. The inflation hub 28 is on the proximal end of the catheter 20 and is used to provide fluid to the lumen 30 for inflating the balloon 24. The inflation hub 28 also provides a grasping location for allowing a user to push the catheter to an appropriate treatment location within a patient's vasculature.

FIG. 2 shows an enlarged view of the distal tip 22 of the catheter 20. The distal tip includes a distal-most end piece 32, a flexible coil section 34 and a safety wire 36. FIG. 2 shows the safety wire 36 connected to the distal-most end piece 32 (e.g., by an adhesive bond or other means). The distal-most end piece 32 forms a smooth, rounded tip that helps guide the catheter 20 through a vessel. The distal-most end piece 32, in addition to providing a smooth rounded tip, connects the distal end of the coil section 34 to the safety wire 36, which traverses the length of the distal tip 22, and is connected to the elongate body 26. The distal safety wire 36 protects the patient in the event the distal tip 22 breaks free of the catheter 20. The distal-most end piece 32 is preferably made of a material such as braze, solder or adhesive.

FIG. 2 shows the coil section 34 of the distal tip 22 including a plurality of continuous helical rings formed by a spiral cut having a varied pitch. It should be noted, however, that this shape is an example only and the distal tip could be any shape, contain different pitches, or have no pitch at all. The depicted cut extends completely through the wall of the tubular elongate body 26. In other embodiments, the cut can be configured so as to not penetrate completely through the wall of the elongate body 26. The cut or cuts can be provided by any number of different techniques including laser cutting, etching, electric discharge machining, etc. Further, the term “cut” will be construed to include terms such as grooves, notches, slots, slits or other terms representative of depressions or openings provided by the removal of material from the elongate body 26. While the distal tip 22 is shown as including continuous helical rings, it will be appreciated that separate discrete cuts such as parallel or angles cuts could also be provided. As depicted, the distal tip 22 is hollow, but could also be solid.

FIG. 3 shows that the coil section 34 of the tip is cut from the elongate body 26. Thus, for example, the coil section 34 is provided by cutting a spiral cut into a solid length of tube defined at the end of the elongate body 26. Thus, the distal tip 22 is an integral part of the elongate body 26. By “integral”, it is meant that the distal tip 22 is unitary or made as a single seamless piece with the elongate body 26. The material used to construct the elongate body 26 and the distal tip 22 of the catheter is, for example, a metal such as nitinol. The distal tip 22 could also be made of, coated with, be impregnated with or otherwise include a radio opaque material. It should be noted, however, that there are many materials that can be used in the present invention and this discussion relates only to the preferred embodiment by example only and in no way limits the teachings of the invention.

By having the distal tip 22 as an integral part of the elongate body 26, a separate bond is not required between the distal tip 22 and the main length of the elongate body 26. By eliminating a bond site on the elongate body 26, a relatively high degree of flexibility can be provided, especially at the distal tip 22. The integral connection also provides a relatively strong connection between the tip and the main portion of the elongate body 26. Moreover, the configuration also assists in maintaining a relatively small diameter. While the integral tip is one inventive aspect disclosed herein, it will be appreciated that other inventive aspects disclosed herein may be practiced with or without a catheter having an integral distal tip.

Referring to FIG. 3, the balloon 24 is shown mounted immediately proximal to the distal tip 22. The balloon 24 has distal and proximal ends 25, 27 that are circumferentially bonded or otherwise connected to the exterior of the elongate member 26. An interior of the balloon 24 is in fluid communication with the lumen 30 of the elongate member 26. For example, ports 38 are shown providing fluid communication between the lumen 30 and the interior of the balloon 24. The balloon 24 can be made of any material conventionally used to make angioplasty balloons. For example, the balloon can be made of a polymeric material such as nylon, PET (Polyethylene Terephthalate or Polyurethane. The balloon is expandable from a deflated diameter (shown by the solid line in FIG. 3) to an expended diameter (shown by the phantom line in FIG. 3). The balloon 24 is inflated by directing fluid through ports 38 into the interior of the balloon 24. The fluid is provided to the ports 38 by injecting fluid into the lumen 30 through the hub 28. The balloon is deflated by withdrawing fluid from the balloon through lumen 30. In one embodiment, the balloon has a deflated outer diameter in the range of 0.025″-0.028″, and an inflated outer diameter in the range of 1.5 mm to 3.0 mm. Preferably, the deflated outer diameter of the balloon is less than 0.028″. More preferably, the deflated outer diameter of the balloon is less than 0.025″.

FIG. 3 shows the safety wire 36 bonded to the elongate body 26 adjacent a proximal safety wire-bonding hole 40. FIG. 3 also illustrates that the preferred embodiment has marker bands 42, which overlap the distal and proximal ends 25, 27 of the balloon 24. The marker bands 42 are preferably made of a relatively dense material that is X-ray visible. These marker bands are used to show where the balloon is placed relative to the area within a patient desired to be treated (e.g., via fluoroscopy or X-ray imaging).

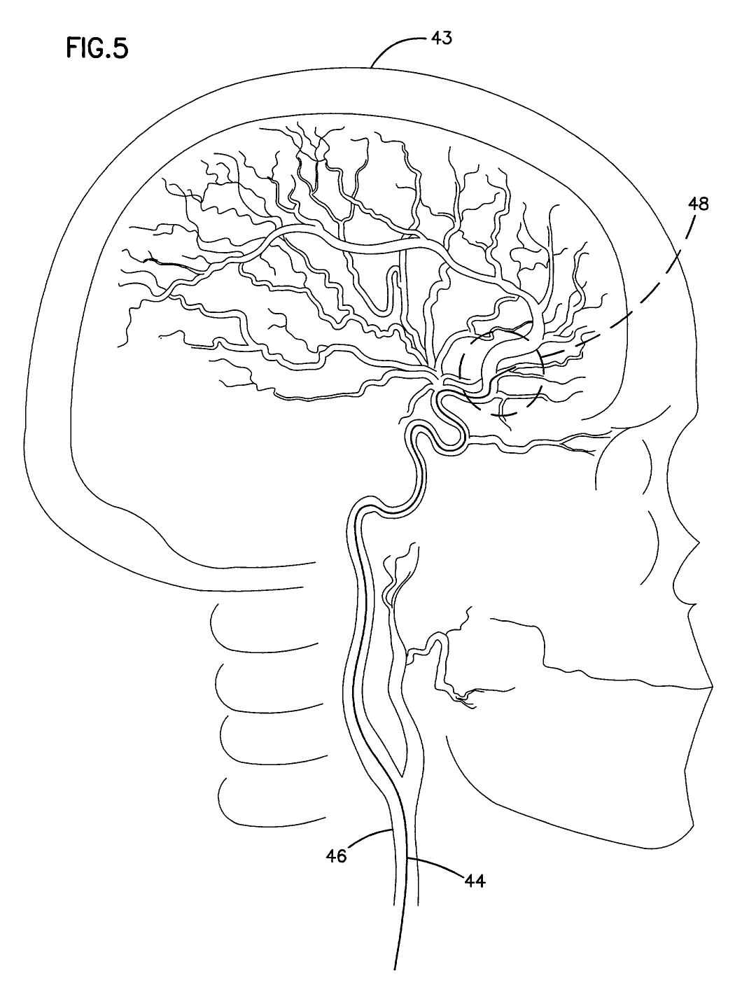

Now referring to FIG. 4, the inflation hub 28 is shown. Inflation hub 24 is connected to the elongate body 26 such that the catheter 20 can be maneuvered through a vessel or guide catheter. Fluids can be delivered by way of the inflation hub through the lumen 30 of the elongate body 26 to the balloon 24. It will be appreciated that the lumen 30 can have any number of different cross-sectional shapes. The lumen 30 is preferably blocked adjacent the balloon 24 to prevent fluids from entering the lumen through the cut of the distal tip 22. For example, a material such as adhesive or other material can be used to provide a fluid barrier between the lumen 30 and the interior of the distal tip 22.

The catheter 20 is preferably sized to allow access to relatively small vessels such as cerebral vessels or vessels of organs such as the liver. Example vessels that may be accessed include the internal carotid artery, the posterior communicating artery, the anterior choroidal artery, the middle cerebral artery, the anterior cerebral artery, the artery of hubner, vertebral artery, the basilar artery, posterior cerebral artery, the posterior choroidal artery, the superior cerebella artery, the anterior inferior cerebella artery, and the posterior inferior cerebella artery. To gain access to these types of vessels, the distal tip 22 preferably has an outer diameter in the range of 0.010″-0.014″. In a preferred embodiment, the distal tip 22 has a maximum outer diameter less than 0.014″. In a more preferred embodiment, the distal tip has a maximum outer diameter less than 0.010″. While the distal tip 22 has been depicted as having a constant outer diameter, the diameter could also be tapered or stepped.

To maintain a small outer diameter, it is preferred for the elongate body 26 to include only one lumen (e.g., the lumen 30 for providing fluid to the balloon). It is contemplated that other embodiments may utilize secondary lumen for receiving guidewires. However, the single lumen configuration is preferred so as to maintain the smallest outer diameter possible. In one embodiment, the lumen has a transverse cross-sectional area in the range of 0.00006-0.00007 IN². This cross-sectional area is preferably large enough to allow for the efficient transfer of fluid to the balloon, but small enough for the catheter to maintain a relatively small outside diameter.

The main body (i.e. the portion proximal to the balloon) of the elongate member 26 also preferably has a relatively small diameter. For example, in one embodiment, the outer diameter of the main body may range from 0.012″ to 0.017″. In certain embodiments, the outer diameter of the elongate member 26 can continuously transition so as to be larger adjacent the proximal end and smaller adjacent the distal end. In other embodiments, the elongate member 26 can include several segments having different outer diameters. In such embodiments, the segments preferably step down to smaller diameters as the elongate member 26 extends toward the distal end. The inner diameter of the elongate member 26 can be constant or can vary. In one embodiment, a wall thickness of the elongate member 26 ranges from .0015″ to .004″. The inner diameter of the elongate body 26 is preferably less than 0.017 inch, more preferably less than 0.01 inch, and most preferably less than 0.005 inch. The outside diameter of the elongate body 26, in one embodiment, ranges from 0.03 inch to 0.025 inch. Preferably, the maximum outer diameter of the elongate body 26 is less than 0.03 inches. More preferably, the maximum outside diameter of the elongate body 26 is less than 0.025 inches.

The catheter 20 is preferably long enough to reach a person's brain when inserted through the patient's femoral artery. In one embodiment, the length of the catheter from the distal-most tip to the base of the hub is in the range of 150 cm.

A lubricious coating such as a hydrophilic coating can be applied or otherwise provided on the exterior surface of the catheter 20 to facilitate insertion into a patient's vasculature. The lubricious coating assists in reducing friction in the vasculature. While a polymeric sheath or other cover could be provided over the elongate body 26, it is preferred to not provide such a sheath so as to maintain a small outer diameter.

FIGS. 5-11 illustrate an example method for using the embodiment of FIGS. 1-4. The method includes the step of inserting a guidewire into a patient and maneuvering it to a treatment area having a small vessel diameter (e.g., a cerebral vessel or a vessel of an organ such as the liver). The method also includes the step of inserting a guide catheter (also referred to as a diagnostic catheter) over the guidewire and maneuvering the guide catheter to the treatment area. The method further includes the step of removing the guidewire from within the guide catheter, and directing a catheter in accordance with the principles of the present invention through the guide catheter to the treatment site for providing treatment (e.g., balloon angioplasty) at the treatment site.

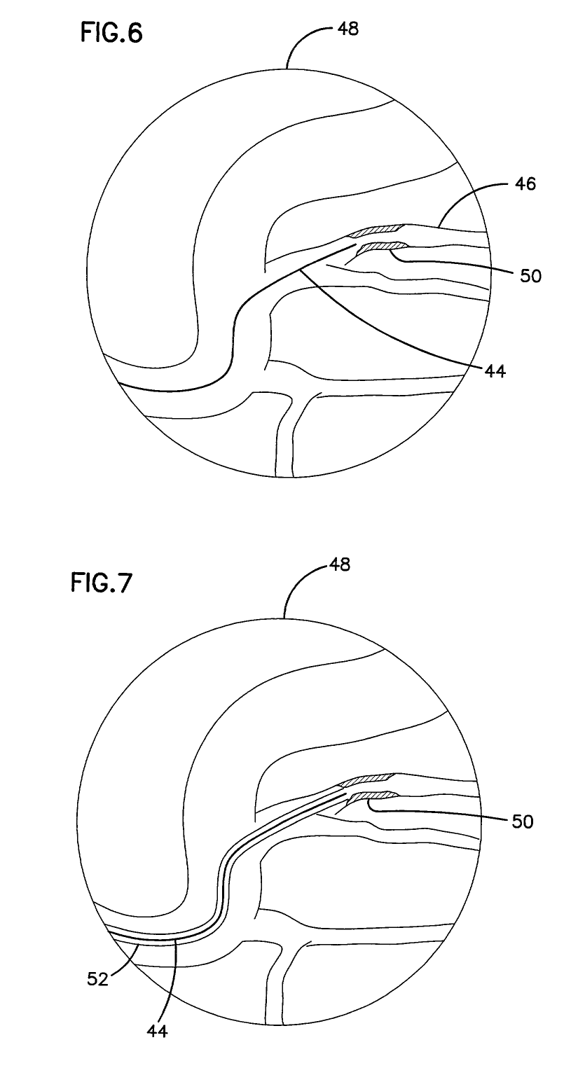

FIG. 5 shows a vascular system of a human brain 43. The vascular system includes a cerebral blood vessel 46 and an occluded vessel 50. A treatment area or site 48 is located adjacent the occlusion of the occluded vessel 50. As shown in FIG. 5, a guidewire 44 is positioned within the blood vessel 46 with a distal end of the guidewire 44 being located at the treatment site 48 adjacent the occlusion of the occluded vessel 50. An enlarged view of the treatment area is shown in FIG. 6.

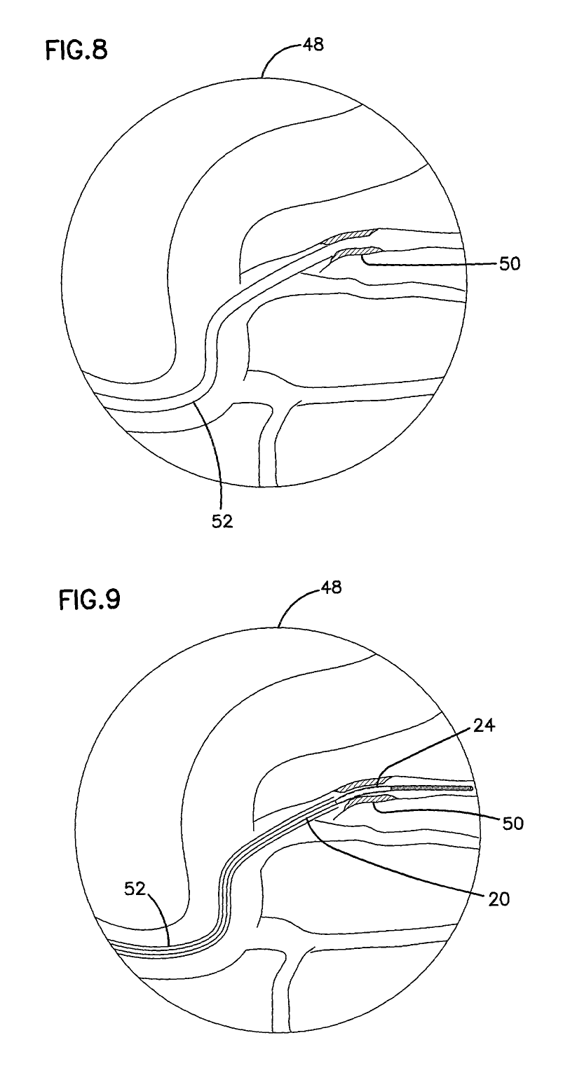

Once the guidewire 44 has been navigated to the treatment area 48 as shown in FIGS. 5 and 6, a guide catheter 52 is preferably pushed over the guidewire 44 until a distal end of the guide catheter 52 is positioned in close proximity to the occlusion of the occluded vessel 50 (see FIG. 7). Once the guide catheter is positioned as shown in FIG. 7, the guidewire 44 can be removed (see FIG. 8) and a diagnostic procedure can be conducted. For example, a radiopaque fluid capable of being seen under fluoroscopy can be injected through the guide catheter 52 to the treatment area 48 such that blood flow at the treatment area 48 can be viewed under fluoroscopy or X-ray. Alternatively, it may be desirable to provide therapeutic drugs, used for clot dissolving such as Urokinase or TPA (Tissue Plasminogen Activator) to the treatment area 48.

After blood flow through the treatment area 48 has been diagnosed and the exact nature of the occlusion determined, the catheter 20 can be inserted through the guide catheter 52 until the distal tip 22 reaches the occlusion of the occluded vessel 50. Once the distal tip 22 is located at the occlusion, the distal tip 22 is preferably forced through the occlusion as shown in FIG. 9 until the balloon 24 is placed inside the occluded area of the occluded vessel 50.

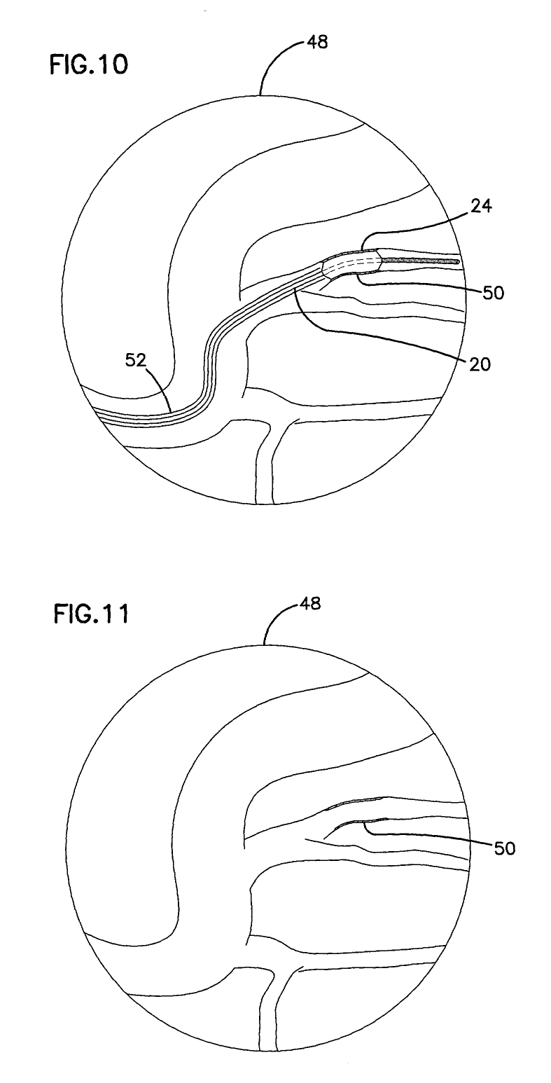

Once the balloon is positioned as shown in FIG. 9, balloon 24 of the catheter 20 can be inflated to enlarge the occluded area by compressing the plaque of the occlusion against the wall of the vessel (see FIG. 10). After the vessel 50 has been opened, the balloon 24 is deflated and removed from the guide catheter 52. With the guide catheter 52 still in place, additional diagnostic procedures such as fluoroscopy or localized drug treatment can be conducted. After the final diagnostic and treatment procedures have been conducted at the treatment area 48, the guide catheter 52 can be removed as shown in FIG. 11.

The above specification provides examples of numerous inventive concepts and features. It will be appreciated that the broad concepts of the present invention are not limited by the specific embodiments shown, but are instead defined by the claims attached hereto. Consequently, the principles of the present disclosure are also applicable to larger vessels, such as coronary vessels.

What is claimed is:

1. A medical device comprising:
an elongate body having distal and proximal ends, the elongate body defining a lumen;
an expandable balloon mounted adjacent the distal end of the elongate body, the balloon being in fluid communication with the lumen of the elongate body; and
a flexible tip located at the distal end of the elongate body that is an integral part of the elongate body.

2. The medical device of claim 1, wherein the flexible tip includes a coil.

3. The medical device of claim 1, wherein the flexible tip includes one or more cuts for providing flexibility.

4. The medical device of claim 1, wherein the elongate body includes metal.

5. The medical device of claim 4, wherein the metal includes Nitinol.

6. The medical device of claim 1, wherein a safety member is connected between a main body of the elongate body and the flexible tip.

7. The medical device of claim 6, wherein the safety member includes a wire attached to a distal-most end of the flexible tip.

8. The medical device of claim 1, wherein the elongate body defines a single lumen.

9. The medical device of claim 1, wherein the flexible tip has a maximum outer diameter less than 0.014″.

10. The medical device of claim 1, wherein the flexible tip includes a radio opaque material.

11. A method for making a medical device comprising:
providing an elongate body having a lumen;
removing a portion of the elongate body adjacent one end of the elongate body to provide a flexible tip; and
attaching an expandable balloon adjacent the flexible tip such that the balloon is in fluid communication with the lumen of the elongate body.

12. A medical device comprising:
an elongate body having distal and proximal ends, the elongate body defining a lumen;
an expandable balloon positioned adjacent the distal end of the elongate body, the balloon having an interior region that is in fluid communication with the lumen; and
a flexible tip located at the distal end of the elongate body, the flexible tip having a maximum outer diameter less than 0.014″ inches.

13. The medical device of claim 12, wherein the flexible tip is integral with the elongate body.

14. The medical device of claim 12, wherein the flexible tip is defined by one or more cuts.

15. The medical device of claim 12, wherein the flexible tip includes a radio opaque material.

16. The medical device of claim 14, wherein the one or more cuts includes a helical cut.

17. The medical device of claim 12, wherein the elongate body includes metal.

18. The medical device of claim 16, wherein the metal includes Nitinol.

19. The medical device of claim 12, wherein the elongate body includes a single lumen.

20. A method for conducting a balloon angioplasty procedure on a cerebral vessel, the method comprising:
inserting a guide catheter into the vasculature of a patient and navigating the guide catheter to an occlusion in the cerebral vessel;
inserting a balloon catheter through the guide catheter and aligning a balloon of the balloon catheter with the occlusion; and
enlarging the occlusion by inflating the balloon.

21. The method of claim 20, further comprising removing the balloon catheter after the occlusion has been enlarged, and conducting diagnostic procedures through the guide catheter.

22. The method of claim 20, further comprising conducting diagnostic procedures through the guide catheter prior to insertion of the balloon catheter.
